(19) United States
(12) Patent Application Publication
(10) Pub. No.: US 2023/0244072 A1
(43) Pub. Date: Aug. 3, 2023

(54) QUANTIFYING EFFECTS OF DOLOMITIZATION ON POROSITY UNDER PETROGRAPHIC MICROSCOPE

(71) Applicant: SAUDI ARABIAN OIL COMPANY, Dhahran (SA)

(72) Inventors: Peng Lu, Dhahran (SA); Yufeng Cui, Beijing (CN); Yin Xu, Dhahran (SA); David Tang, Dhahran (SA)

(73) Assignees: SAUDI ARABIAN OIL COMPANY, Dhahran (SA); Aramco Far East (Beijing) Business Services Co., Ltd., Beijing (CN)

(21) Appl. No.: 17/649,501

(22) Filed: Jan. 31, 2022

(51) Int. Cl. G02B 21/36; G01N 33/24; G06T 7/00

(52) U.S. Cl. CPC G02B 21/365; G01N 33/24; G06T 7/0002; G06T 2207/10024; G06T 2207/10056; G06T 2207/30181

(57) ABSTRACT

A method for evaluating reservoir quality of a carbonate rock includes: acquiring a thin section of the carbonate rock with a predetermined thickness; obtaining a microscopic image of the thin section; analyzing the microscopic image of the thin section to determine a content of one or more components of the carbonate rock selected from a group of calcite, pore space, replacive dolomite, dolomite cement, and matrix; and calculating a change of porosity based on the content of the one or more components.

### BACKGROUND

About half of oil and gas reservoirs around the world is composed of carbonate rocks. Two major components of carbonate rocks are limestone which is composed of calcite and dolomite rock which is composed of mineral dolomite. Dolomite, with the formula CaMg(CO₃)₂, may be formed through two diagenetic processes: dolomitization, which is the replacement of calcite by dolomite, and dolomite cementation (or overdolomitization), which is the precipitation of dolomite from fluids as a cement in pore space.

The dolomite formation processes may strongly influence the redistribution of pore structure and connectivity, permeability pathway, and reservoir producibility, because dolomite may behave as either a conduit or a barrier to a flow depending on its composition and property from different formation processes. For example, dolomitization and dolomite cementation may have different effects on change of porosity, where replacive dolomite originated via replacement of calcite causes an increase in porosity and dolomite cement formed in dolomite cementation causes a decrease in porosity.

Therefore, studies on dolomite formation processes are of great importance in understanding and predicting the reservoir quality, especially porosity and permeability, of carbonate reservoirs.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to a method for evaluating reservoir quality of a carbonate rock. The method comprises: acquiring a thin section of the carbonate rock with a predetermined thickness; obtaining a microscopic image of the thin section; analyzing the microscopic image of the thin section to determine a content of one or more components of the carbonate rock selected from a group of calcite, pore space, replacive dolomite, dolomite cement, and matrix; and calculating a change of porosity based on the content of the one or more components. The microscopic image may be an optical image under a plain polarized light or a cathodoluminescence image. The method further comprises staining the thin section with a red dye, a blue dye, or both. The analyzing process may include point counting of at least 200 points, wherein the content of the one or more components in the thin section is proportional to a number of points having corresponding texture or color in the microscopic image. The point counting may be performed from 400 to 600 points. The point counting may base on different textures or colors of replacive dolomite and dolomite cement in a cathodoluminescence image. The method may further comprise determining the reservoir quality, including properties of porosity and permeability, based on the change of porosity.

In another aspect, embodiments disclosed herein relate to a system for evaluating reservoir quality of a carbonate rock comprising: a microscope configured to obtain a microscopic image of a thin section of the carbonate rock; and a processor configured to analyze the microscopic image of the thin section to determine a content of one or more components of the carbonate rock selected from a group of calcite, pore space, replacive dolomite, dolomite cement, and matrix, and calculate a change of porosity based on the content of the one or more components. The microscopic image may be an optical image under a plain polarized light or a cathodoluminescence image. The thin section of the carbonate rock may be stained with a red dye, a blue dye, or both. The processor may be configured to analyze the microscopic image by point counting of at least 200 points, wherein the content of the one or more components in the thin section is proportional to a number of points having corresponding texture or color in the microscopic image. The processor may be configured to analyze the microscopic image by point counting of from 400 to 600 points. The processor may be configured to perform point counting based on different textures or colors of replacive dolomite and dolomite cement in a cathodoluminescence image. The processor may be configured to determine the reservoir quality, including properties of porosity and permeability, based on the change of porosity.

Other aspects and advantages of the claimed subject matter will be apparent from the following description and the appended claims.

### BRIEF DESCRIPTION OF DRAWINGS

FIG. 1 shows a method for evaluating reservoir quality of a carbonate rock according to one or more embodiments of the present invention.

FIG. 2A shows an optical image of a thin section of carbonate rock according to the method of one or more embodiments.

FIG. 2B shows a cathodoluminescence image of a thin section according to the method of one or more embodiments.

FIG. 2C shows point counting of a cathodoluminescence image of a thin section according to the method of one or more embodiments.

FIG. 3A shows an optical image of a thin section of carbonate rock used according to the method of one or more embodiments.

FIG. 3B shows a cathodoluminescence image of a thin section according to the method of one or more embodiments.

FIG. 3C shows point counting of a cathodoluminescence image of a thin section according to the method of one or more embodiments.

### DETAILED DESCRIPTION

In the following, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Carbonate rocks are mainly composed of calcite and dolomite, with a minority of non-carbonate minerals including anhydrite and silicate minerals (for example, quartz, feldspar, and clay minerals). Dolomite is mainly formed through two diagenesis processes: dolomitization represented by Equation 1 and dolomite cementation represented by Equation 2.

$$2 {\text{CaCO}}_{3} \left(\text{calcite}\right) + {\text{Mg}}^{2 +} \to \text{CaMg} {\left({\text{CO}}_{3}\right)}_{2} \left(\text{dolomite}\right) + {\text{Ca}}^{2 +}$$

$${\text{Ca}}^{2 +} + {\text{Mg}}^{2 +} + 2 {\text{CO}}_{3} {}^{2 \text{-}} \to \text{CaMg} {\left({\text{CO}}_{3}\right)}_{2} \left(\text{dolomite}\right)$$

Dolomitization is a geological process by which the carbonate mineral dolomite is formed when magnesium ions replace calcium ions in another carbonate mineral, calcite, partially or entirely under a wide range of crystallization temperatures and fluids. Dolomitization may cause an increase in porosity. Based on the volume of two moles of calcite (molar volume of 36.934 cm³/mol) and the volume one mole of dolomite (molar volume of 64.365 cm³/mol), the replacement of calcite results in a net decrease of 9.503 cm³/mol in volume, which in turn increases porosity by about 12.3%. By determining the amount of replacive dolomite, it is possible to calculate the contribution of dolomitization to the change of porosity.

Dolomite cementation may persist due to continuous supply of dolomitizing fluids. Dolomite cementation is precipitation of ions in the dolomitizing fluids to form dolomite cement having a molar volume of 64.365 cm³/mol. The increase in volume of the dolomite cement results in blocking of the pore space and thus a decrease in porosity.

To better understand and predict the reservoir quality, especially the porosity and permeability, one or more embodiments of the present invention provide a method for quantifying the effects of different dolomite formation processes in change of porosity. The method determines the content of at least one of calcite, dolomite (including replacive dolomite and dolomite cement), and pore space in a carbonate rock and quantifies the effects of dolomitization and/or dolomite cementation to the change of porosity based on the content of each component.

In one or more embodiments, the method of the present invention comprises acquiring a thin section of the carbonate rock with a predetermined thickness. The thin section may be polished or unpolished. A thin piece with suitable size may be cut from a large piece of rock with a diamond saw and ground optically flat. The thin piece is then mounted on a glass slide and ground smooth using progressively finer abrasive grit until the sample is at a thickness of interest. The thickness of the thin piece may range from about 20 to about 100 µm, or may be 30 µm, or may be 40 µm. A Michel-Lévy interference color chart may be used to determine the thickness, optionally using quartz as a thickness gauge.

In one or more embodiments, the method of the present invention comprises staining the carbonate rock with at least a red dye. Calcite and dolomite are both gray under plain polarized light before staining but may be distinguishable after staining with the red dye. The red dye used in staining may be any suitable dye known to one skilled in the art, such as potassium ferricyanide and Alizarin red. Calcite is stained by the red dye to a red color, while dolomite is not stained and remains its original color. Staining the carbonate rock enables recognition of calcite.

In one or more embodiments, the method of the present invention comprises staining the carbonate rock with at least a blue dye. The carbonate rock may be injected with an epoxy resin with a blue dye or a fluorescent epoxy that is excited by blue light. Pore spaces may be filled with the blue dye to clarify porosity and pore geometries. The blue dye may be any suitable dye known to one skilled in the art, such as Orasol Blue 825. In one or more embodiments, the blue dye may be used to automatically or manually identify pores and characterize pore properties.

In one or more embodiments, the method of the present invention comprises obtaining an optical image of the carbonate rock. An optical microscope may be used to generate magnified optical images of a thin section of the carbonate rock. The microscope may be in transmission mode or in reflective mode, bright field or dark field, under polarized light or unpolarized light. In one or more embodiments, the optical microscope is a petrographic microscope, which may include one or more of polarizing filter, rotating stage, phase telescope, and wave plate to facilitate study of minerals or rocks whose optical properties can vary with orientation. The petrographic microscope is used to identify rocks and minerals in thin sections (i.e., thin slices of a rock, mineral, or the like) and provides information on detailed descriptions of rocks and minerals.

In one or more embodiments, the method of the present invention comprises obtaining a cathodoluminescence image of the carbonate rock. At least replacive dolomite formed by dolomitization and dolomite cement formed by dolomite cementation are distinguishable in the cathodoluminescence image. Cathodoluminescence provides information on spatial distribution of trace elements, particularly Fe²⁺ and Mn²⁺, in calcite, dolomite formed by different processes, and other grains, therefore may be used to map the various zones and their relative timing and origin. According to one or more embodiments, the cathodoluminescence image of carbonate rocks may be obtained through a petrographic microscope functioned with a cathodoluminescence attachment. The cathodoluminescence attachment uses an electron source (e.g., a cathode gun) to bombard a thin section of carbonate rock with a beam of high-energy electrons. Subsequent transition from an excited state to a normal state causes the emission of light (luminescence) that can be observed in various colors and intensities using a polarizing microscope. The resulting luminescence in minerals provides information on textures and compositional variations that are not otherwise evident under plain unpolarized or polarized light. Internal structures of minerals become visible and are indicative of growth zoning, trace element zoning, and structures allowing processes such as mineral growth, resolution, and alteration to be reconstructed. The cathodoluminescence attachment to a microscope allows a sample such as thin sections of the carbonate rock to be examined optically with the microscope and with cathodoluminescence in the same area of interest.

According to one or more embodiments, the method of the present invention comprises analyzing a plurality of points in an optical or cathodoluminescence image to quantitatively determine a content of at least one component in the carbonate rock. For example, the content of calcite may be determined by quantifying zones in the optical image where calcite is stained with a red dye. The content of pore spaces may be determined by quantifying zones in the optical image where pore spaces are stained with a blue dye. The content of pore spaces may also be determined by quantifying zones in the cathodoluminescence image where pore spaces generally show a dark to black color, whether stained or not. Matrix in carbonate, such as carbonate mud, micrite, and microspar, may show a dark to black color in both the optical image and the cathodoluminescence image, and is distinguishable from pore spaces and other components. In one or more embodiments, replacive dolomite and dolomite cement may show different textures and/or different colors in the cathodoluminescence image of the carbonate rock, which may facilitate in distinguishing replacive dolomite and dolomite cement cathodoluminescent zones.

According to one or more embodiments, replacive dolomite and dolomite cement may exhibit different textures in a cathodoluminescence image. Replacive dolomite may show fabric destructive textures at cloudy cores at a center of each grain. Dolomite cement may present as clear rims in forms of well-developed rhombohedral crystals, sharp edges and lamellar twins. Dolomite cement may encompass and circulate the cloudy cores, fill pore spaces or fractures, and occlude porosity. Dolomite cement may have a crystal size varying from fine to coarse at micrometer level. Dolomite cement may form one thin rim overgrowth layer, or two rim overgrowth layers, or three and more rim overgrowth layers.

According to one or more embodiments, replacive dolomite and dolomite cement may exhibit different colors in a cathodoluminescence image. In general, incorporation of Mn²⁺ into the carbonate lattice stimulates luminescence and incorporation of Fe²⁺ reduces or quenches luminescence. In other words, component with a high Mn²⁺/ Fe²⁺ ratio may show a bright luminescence. Because replacive dolomite and dolomite cement are formed at different stages, their contents of trace elements (e.g., Fe²⁺ and Mn²⁺) are different. As a result, replacive dolomite and dolomite cement may show different colors in the cathodoluminescence image, with information on spatial distribution of each component. In one or more embodiments, replacive dolomite may show gray in color under plane polarized light and exhibit an overall homogenous dull red luminescence in the cathodoluminescence image. Dolomite cement may show light gray in color under plane polarized light and a bright red luminescence in the cathodoluminescence image. While the colors of replacive dolomite and dolomite cement may not be distinguishable under plain unpolarized light, they may show different colors in the cathodoluminescence image. It is noted that dolomite cement does not necessarily show a bright red luminescence. In one or more embodiments, replacive dolomite may show a dull red luminescence and dolomite cement may show a bright red luminescence.

According to one or more embodiments, the method of the present invention comprises analyzing an optical or cathodoluminescence image of the carbonate rock to quantify the content of each component. As previously described, calcite, dolomite (replacive dolomite and dolomite cement), and pore space may be distinguished under microscopy, and the content of each component in the carbonate rock may be quantitatively estimated through point counting. Point counting is a technique used in geology capable of statistically measuring the content of each component in the carbonate rock. In one or more embodiments, more than 200 points, or more than 300 points, or more than 400 points, or 200 to 1000 points, or 200 to 800 points, or 400 to 600 points are randomly picked and counted in each cathodoluminescence image. Based on the point counting results, weight percentage of each component in the carbonate rock, which is proportional to the numbers of points for each component, is obtained.

In one or more embodiments, the method of the present invention comprises calculating a change of porosity based on the result of point counting. The calculation may base on the content of each component in the carbonate rock. For example, the change of porosity may be calculated using Equation 3 based on the volume change of porosity caused by replacive dolomite formed by dolomitization and dolomite cement formed by dolomite cementation.

$$\begin{matrix}\text{Change of porosity} \text{=} \text{Dolomite cement} \left(\text{\%}\right) - \left(\text{9} \text{.503} / \text{64} \text{.365}\right) \text{×} \\ \text{Replacive dolomite} \left(\text{\%}\right)\end{matrix}$$

In one or more embodiments, the change of porosity caused by dolomitization and dolomite cementation may be used to evaluate an original porosity of the carbonate rock, represented by Equation 4.

$$\text{Original porosity} = \text{Measured porosity + Change of porosity}$$

The measured porosity may be obtained based on point counting results of pore spaces which can be dyed to blue or show a dull red luminescence under cathodoluminescence. The change of porosity may be calculated by point counting results of replacive dolomite and dolomite cement based on the cathodoluminescence image of the carbonate rock. The original porosity of the carbonate rock provides useful information in understanding and predicting the reservoir quality of the carbonate rock. The original porosity, final measured porosity, and change of porosity may provide useful information in understanding and predicting the reservoir quality of the carbonate rock.

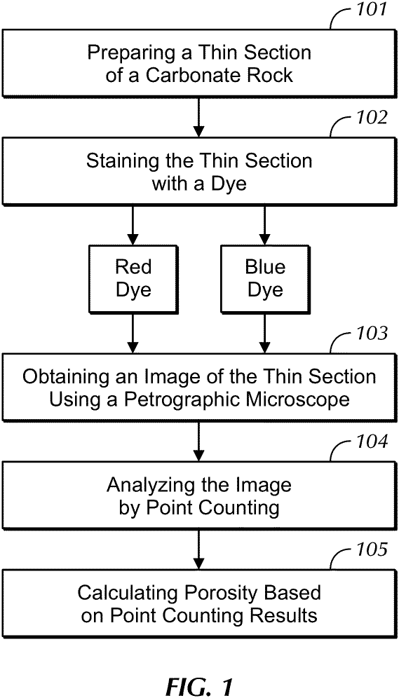

The method according to one or more embodiments of the present invention is shown in FIG. 1. In step 101, a thin section of a carbonate rock to be analyzed is prepared. The thin section may be a thin slice of the carbonate rock, grounded, polished or unpolished. In step 102, the thin section is stained with at least a dye. The dye may be a red dye and/or a blue dye. The red dye is used for recognition of calcite rather than dolomite and the blue dye is used for recognition of pore spaces. In step 103, an image of the thin section of the carbonate rock is obtained by a petrographic microscope. The image may be an optical image under plain polarized light and/or a cathodoluminescence image. In step 104, the image of the thin section of the carbonate rock is analyzed to obtain quantitative information of one or more component in the carbonate rock. The analysis may be performed by point counting, based on different textures and/or different colors of each component in the image. For example, calcite may be dyed to red and pore spaces may be dyed to blue. Thus, point counting results of red calcite and blue pore spaces may represent a percentage of calcite and pore spaces in the thin section. Replacive dolomite and dolomite cement are not distinguishable under plain polarized light, but are evident in the cathodoluminescence image. Replacive dolomite and dolomite cement may show different textures and/or different colors represented by different colors in red luminescence. The pore spaces dyed to blue may show a dark or black color in the cathodoluminescence image. At least 200 points may be used in point counting for quantification of at least one of calcite, pore spaces, replacive dolomite, and dolomite cement. Point counting results of replacive dolomite and dolomite cement provides quantitative effect of dolomitization and dolomite cementation processes in the formation of the carbonate rock. In step 105, a calculation is performed to obtain information regarding a change of porosity and/or an original porosity of the thin section of the carbonate rock. The calculation may be performed according to one or more embodiments described above. The calculation results may be used to determine the reservoir quality of the carbonate rock.

In one or more embodiments, at least one or more step in the method disclosed herein is realized by a computer, which may be any suitable computer including commercially available desktop and laptop computers loaded with and running appropriate software. The computer, data collection and processing, point counting, and calculations of the embodiments described herein may be performed using hardware and software to implement the various modules, elements, components, methods, and algorithms described herein, and can include a processor configured to execute one or more sequences of instructions, programming stances, or code stored on a non-transitory computer-readable medium. The processor can be, for example, a general purpose microprocessor, a microcontroller, a digital signal processor, an application specific integrated circuit, a field programmable gate array, a programmable logic device, a controller, a state machine, a gated logic, discrete hardware components, an artificial neural network, or any like suitable entity that can perform calculations or other manipulations of data. In some embodiments, computer hardware can further include elements such as, for example, a memory (e.g., random access memory (RAM), flash memory, read only memory (ROM), programmable read only memory (PROM), erasable programmable read only memory (EPROM)), registers, hard disks, removable disks, CD-ROMS, DVDs, or any other like suitable storage device or medium.

Executable sequences described herein can be implemented with one or more sequences of code contained in a memory. In some embodiments, such code can be read into the memory from another machine-readable medium. Execution of the sequences of instructions contained in the memory can cause a processor to perform the process steps described herein. One or more processors in a multi-processing arrangement can also be employed to execute instruction sequences in the memory. In addition, hard-wired circuitry can be used in place of or in combination with software instructions to implement various embodiments described herein. Thus, the present embodiments are not limited to any specific combination of hardware and/or software.

As used herein, a machine-readable medium will refer to any medium that directly or indirectly provides instructions to a processor for execution. A machine-readable medium can take on many forms including, for example, non-volatile media, volatile media, and transmission media. Non-volatile media can include, for example, optical and magnetic disks. Volatile media can include, for example, dynamic memory. Data transmission media can include, for example, coaxial cables, wire, fiber optics, and wires that form a bus. Common forms of machine-readable media can include, for example, floppy disks, flexible disks, hard disks, magnetic tapes, other like magnetic media, CD-ROMs, DVDs, other like optical media, punch cards, paper tapes and like physical media with patterned holes, RAM, ROM, PROM, EPROM and flash EPROM.

### EXAMPLES

### Example 1

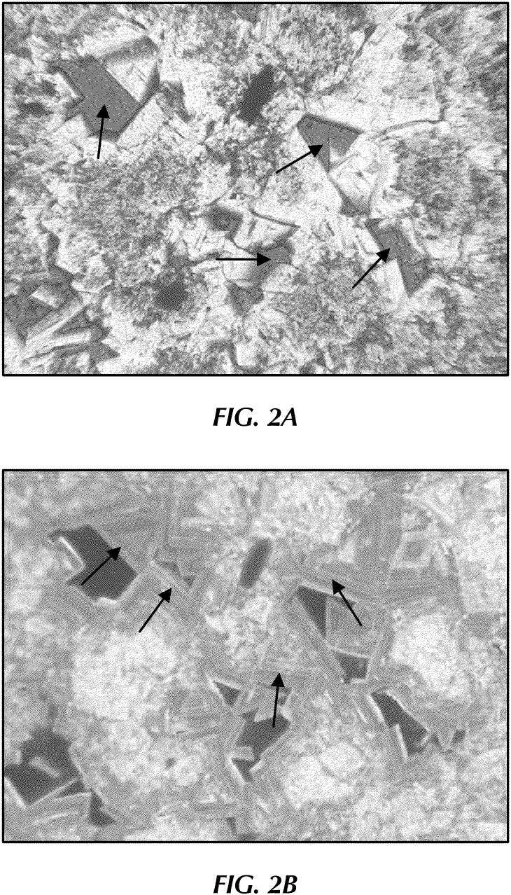

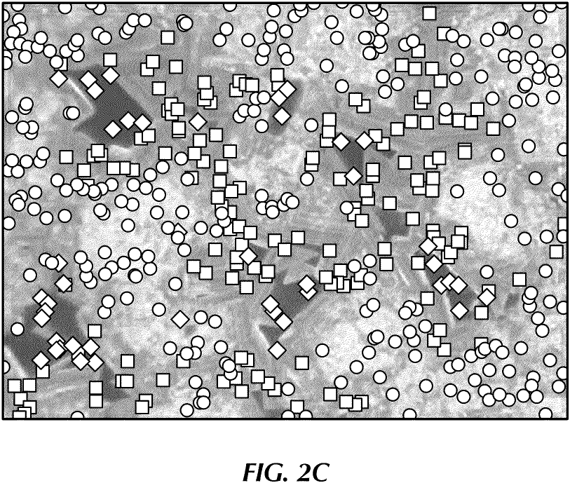

A thin section of a carbonate rock was used as sample #1 for analysis of porosity. Sample #1 was stained by Alizarin Red S in a 0.2% HCl solution, causing calcite in the carbonate rock to turn red, whereas dolomite remained its original color. Sample #1 was also stained by a blue dye for recognition of pore spaces. A petrographic microscope functioned with cathodoluminescence attachment was used to obtain both an optical image under plain polarized light and a cathodoluminescence image of sample #1, as shown in FIGS. 2A and 2B. In the optical image, dolomite showed its original color and pore spaces were dyed to blue, as indicated by arrows. In the cathodoluminescence image, replacive dolomite and dolomite cements were shown as fabric-destructive textures at cloudy cores and well-developed rhombohedral crystals as clear rims, respectively. The single to multiple, thin to thick cathodoluminescent zones at the rims are indicated by arrows.

A point counting method was applied to the cathodoluminescence image to quantify each component in the carbonate rock sample #1. 500 points were used to determine the percentage of replacive dolomite, dolomite cement, and pore space. The results of point counting are shown in FIG. 2C, indicating that sample #1 contained 55.8 % of replacive dolomite (indicated by circles), 35.6 % of dolomite cement (indicated by squares), and 8.6 % of pore space (indicated by diamonds). A change of porosity was determined to be 27.36 % based on Equation 3 and the original porosity of sample #1 was calculated to be 35.96 % based on Equation 4.

### Example 2

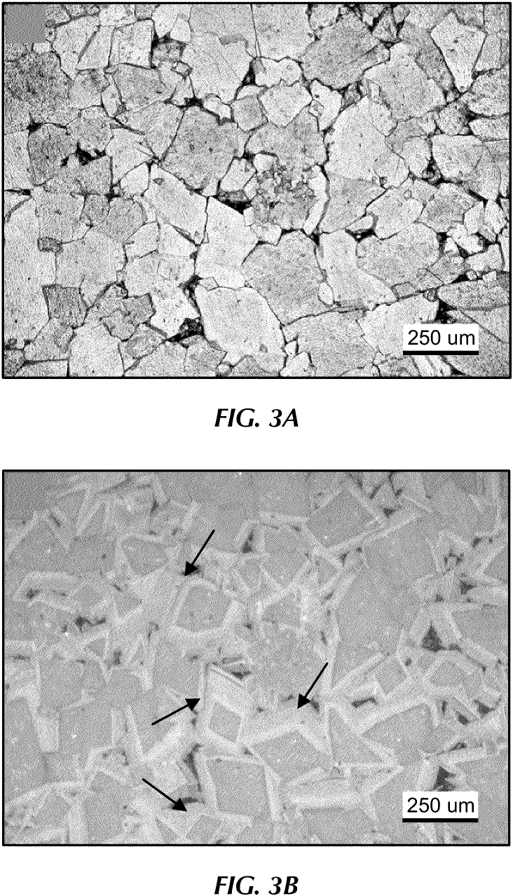

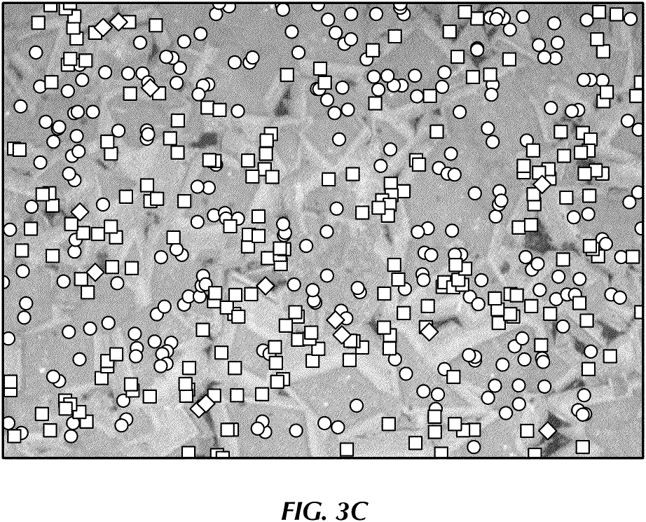

Another thin section of a carbonate rock was used as sample #2 for analysis of porosity. Sample #2 was stained by Alizarin Red S in a 0.2% HCl solution, causing calcite in the carbonate rock to turn red, whereas dolomite remained its original color. A petrographic microscope functioned with cathodoluminescence attachment was used to obtain both an optical image under plain polarized light and a cathodoluminescence image of sample #2, as shown in FIGS. 3A and 3B. In the optical image, no visible pore space was observed. In the cathodoluminescence image, replacive dolomite was fabric-destructive with cloudy cores and replacive dolomite was characterized by euhedral to subhedral dolomite crystals as clear crystal rims. A unimodal crystal size distribution ranged from about 50 to about 250 µm. The cathodoluminescence image of the carbonate rock displayed a dull luminescence core with single to multiple, thin to thick rims with bright luminescence.

A point counting method was applied to the cathodoluminescence image to quantify each component in carbonate rock sample #2. 500 points were used to determine the percentage of replacive dolomite, dolomite cement, and matrix, based on different colors. The results of point counting are shown in FIG. 3C, indicating that sample #2 contained 50.6 % of replacive dolomite (indicated by circles), 46.6 % of dolomite cement (indicated by squares), and 2.8 % of matrix (indicated by diamonds). A change of porosity was determined to be 39.13 % based on Equation 3 and the original porosity of sample #2 was calculated to be 39.13 %, since measured porosity is considered to be 0 when no visible pore space is observed from the optical image.

Unless defined otherwise, all technical and scientific terms used have the same meaning as commonly understood by one of ordinary skill in the art to which these systems, apparatuses, methods, processes and compositions belong.

The ranges of this disclosure may be expressed in the disclosure as from about one particular value, to about another particular value, or both. When such a range is expressed, it is to be understood that another embodiment is from the one particular value, to the other particular value, or both, along with all combinations within this range.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed:

1. A method for evaluating reservoir quality of a carbonate rock comprising:
acquiring a thin section of the carbonate rock with a predetermined thickness;
obtaining a microscopic image of the thin section;
analyzing the microscopic image of the thin section to determine a content of one or more components of the carbonate rock selected from a group of calcite, pore space, replacive dolomite, dolomite cement, and matrix; and
calculating a change of porosity based on the content of the one or more components.

2. The method of claim 1, wherein the microscopic image is an optical image under a plain polarized light.

3. The method of claim 1, wherein the microscopic image is a cathodoluminescence image.

4. The method of claim 1, further comprises staining the thin section with a red dye.

5. The method of claim 1, further comprises staining the thin section with a blue dye.

6. The method of claim 1, wherein the analyzing includes point counting of at least 200 points,
wherein the content of the one or more components in the thin section is proportional to a number of points having corresponding texture or color in the microscopic image.

7. The method of claim 6, wherein the analyzing includes point counting of from 400 to 600 points.

8. The method of claim 6, wherein the point counting is based on different textures or colors of replacive dolomite and dolomite cement in a cathodoluminescence image.

9. The method of claim 1, further comprises determining the reservoir quality, including properties of porosity and permeability, based on the change of porosity.

10. A system for evaluating reservoir quality of a carbonate rock comprising:
a microscope configured to obtain a microscopic image of a thin section of the carbonate rock; and
a processor configured to analyze the microscopic image of the thin section to determine a content of one or more components of the carbonate rock selected from a group of calcite, pore space, replacive dolomite, dolomite cement, and matrix, and calculate a change of porosity based on the content of the one or more components.

11. The system of claim 10, wherein the microscopic image is an optical image under a plain polarized light.

12. The system of claim 10, wherein the microscopic image is a cathodoluminescence image.

13. The system of claim 10, wherein the thin section of the carbonate rock is stained with a red dye.

14. The system of claim 10, wherein the thin section of the carbonate rock is stained with a blue dye.

15. The system of claim 10, wherein the processor is configured to analyze the microscopic image by point counting of at least 200 points, wherein the content of the one or more components in the thin section is proportional to a number of points having corresponding texture or color in the microscopic image.

16. The system of claim 15, wherein the processor is configured to analyze the microscopic image by point counting of from 400 to 600 points.

17. The system of claim 15, wherein the processor is configured to perform point counting based on different textures or colors of replacive dolomite and dolomite cement in a cathodoluminescence image.

18. The system of claim 10, wherein the processor is configured to determine the reservoir quality, including properties of porosity and permeability, based on the change of porosity.
